(19) United States
(12) Patent Application Publication
(10) Pub. No.: US 2007/0055073 A1
(43) Pub. Date: Mar. 8, 2007

(54) Methods and apparatus for producing lower alkyl esters

(76) Inventor: Thomas Barton McGurk, Marietta, GA (US)

Correspondence Address: Thomas B. McGurk, 1501 Paxford Overlook, Marietta, GA, 30066, US

(21) Appl. No.: 11/517,168

(22) Filed: Sep. 7, 2006

Related U.S. Application Data
(60) Provisional application No. 60/715,040, filed on Sep. 8, 2005.

(51) Int. Cl. C07C 51/43

(52) U.S. Cl. 554/174

(57) ABSTRACT

Methods and apparatus for producing lower alkyl esters is disclosed in which a reactant mixture is reacted in a dynamic reactor to produce the lower alkyl esters. The reaction takes place within a reaction zone of the dynamic reactor during a mean residence time less than what is generally known in the art in order to produce a product mixture containing primarily lower alkyl esters.

### RELATED APPLICATIONS

This Application claims priority to U.S. Provisional application U.S. Ser. No. 60/715,040, filed on Sep. 8, 2005, which is incorporated by reference as if set forth herein in its entirety.

### TECHNICAL FIELD

The disclosure relates to methods and apparatus for producing esters, particularly lower alkyl esters such as fatty acid alkyl esters.

Demand for lower alkyl esters, particularly fatty acid alkyl esters that meet the requirements necessary to be sold as biodiesel, is high and continuing to grow rapidly. Currently, the demand for such esters is far outpacing present supply. In fact, it is projected that biodiesel demand and production will grow over ten fold over the next five years. Therefore, there is a need for methods and apparatus for producing fatty acid alkyl esters that can help meet this demand.

### SUMMARY

The present disclosure encompasses both methods and systems for producing esters. In particular, methods and systems for producing lower alkyl esters, such as fatty acid alkyl esters are described herein.

In one aspect, the disclosure encompasses a method for producing lower alkyl esters that includes reacting an oil with an alcohol and a catalyst for a mean residence time of less than about one minute in a dynamic reactor to produce a product mixture, wherein in the product mixture contains primarily lower alkyl esters. The product mixture can comprise a majority by weight of lower alkyl esters. The method also can encompass such product mixtures having a conversion rate of glycerides to lower alkyl esters of about 90% or greater. Additionally, such a product mixture can have a conversion rate of about 95% or greater. Furthermore, such product mixture can have a conversion rate of about 99% or greater.

In another aspect, a method for producing an alkyl ester is provided that includes combining an alcohol, a catalyst and an oil to produce a reactant mixture, introducing the reactant mixture into a cavitation zone, reacting the reactant mixture within the cavitation zone to produce a product mixture, separating the product mixture into a light liquid phase and a light heavy phase, wherein the light liquid phase contains primarily lower alkyl esters. The light liquid phase can comprise a majority by weight of lower alkyl esters. Additionally, the method can include separating the product mixture into a vapor product phase and a liquid product phase, wherein the liquid product phase of the product mixture then is separated into the light liquid phase and the heavy liquid phase.

In a further aspect, the disclosure encompasses a method of producing an alkyl ester that includes introducing at least one continuous flow of at least one reactant into a dynamic reactor, reacting the reactants during a mean residence time of about one minute or less in the dynamic reactor to produce a product mixture containing alkyl esters and glycerol, and continuously discharging the product mixture from the dynamic reactor.

In another aspect, a method is disclosed that includes introducing an alcohol, a catalyst and an oil into a dynamic reactor to form a reaction mixture, reacting the reactant mixture in the dynamic reactor to produce a product mixture, separating the product mixture into a light liquid phase and a heavy liquid phase, and continuously reintroducing at least a portion of the light liquid phase into the dynamic reactor along with additional reactant mixture.

In another aspect, a method for producing fatty acid methyl esters is set forth that includes introducing at least one continuous flow of an oil, an alcohol and a catalyst into a dynamic reactor to form a reactant mixture, transesterifying the reactant mixture within the dynamic reactor to produce a product mixture within a mean residence time of about one minute or less, continuously discharging the product mixture from the dynamic reactor, separating vapor from the product mixture, and centrifugally separating the product mixture into a light liquid phase and a heavy liquid phase.

In yet another aspect, a method for producing lower alkyl esters is disclosed that includes combining an alcohol, a catalyst and a biologically derived oil to form a reaction mixture, continuously feeding the reaction mixture into a dynamic reactor, reacting the reaction mixture in the dynamic reactor during a mean residence time of less than about one minute to form a product mixture, continuously discharging the product mixture from the dynamic reactor, separating vapor from the product mixture, and separating the product mixture into at least two phases, one of which contains primarily lower alkyl esters.

In another aspect, a system for producing lower alkyl esters is disclosed that includes a dynamic reactor having an inlet and an outlet. A vapor-liquid separator is in fluid communication with the outlet of the dynamic reactor and is disposed in line between the dynamic reactor and a liquid-liquid separator. The system also includes an oil storage container and an alcohol storage container in fluid communication with the inlet of the dynamic reactor. The system also can include a mixing unit disposed in line between the oil and alcohol storage containers and the inlet of the dynamic reactor. In another aspect, the system can include a heat exchanger operably connected to a conduit for transferring oil to the inlet of the dynamic reactor.

In still a further aspect, the system can include a mixing unit for combining a catalyst component with an alcohol. In one aspect, the mixing unit can be operably connected to a solids or liquid transporter for introducing solid catalyst components to the mixing unit.

In another aspect, the system's mixing unit can include a series of mixing units for combining a catalyst component with an alcohol, wherein each one of the mixing units is optionally in fluid communication with the first reactor.

In yet another aspect, the liquid-liquid separator can include a liquid-liquid-solid separator. In another aspect, the system can include one or more condensers in fluid communication with an outlet of the vapor-liquid separator. In still another aspect, the dynamic reactor can include a cavitation reactor.

In still a further aspect, the disclosure includes a method for producing lower alkyl esters including introducing an oil, a catalyst and an alcohol to a dynamic reactor to produce a reactant mixture that is substantially free of lower alkyl esters, reacting the oil, the catalyst and the alcohol in the dynamic reactor to produce a product mixture, wherein the product mixture contains a majority by volume of lower alkyl esters and wherein the mean residence time of the dynamic reactor is up to about ten seconds. The product mixture also may contain a majority by weight of lower alkyl esters. The method also may include reacting the oil, the catalyst and the alcohol by inducing cavitation therein.

These and other aspects, embodiments and features of the disclosed methods and apparatus are set forth in greater detail below and within the accompanying drawings which are briefly described as follows.

### BRIEF DESCRIPTION OF THE DRAWINGS

FIG. 1 illustrates an apparatus for producing lower alkyl esters.

FIG. 2 illustrates another apparatus for producing lower alkyl esters.

FIG. 3 illustrates yet another apparatus for producing lower alkyl esters.

FIG. 4 illustrates still a further apparatus for producing lower alkyl esters.

### DETAILED DESCRIPTION

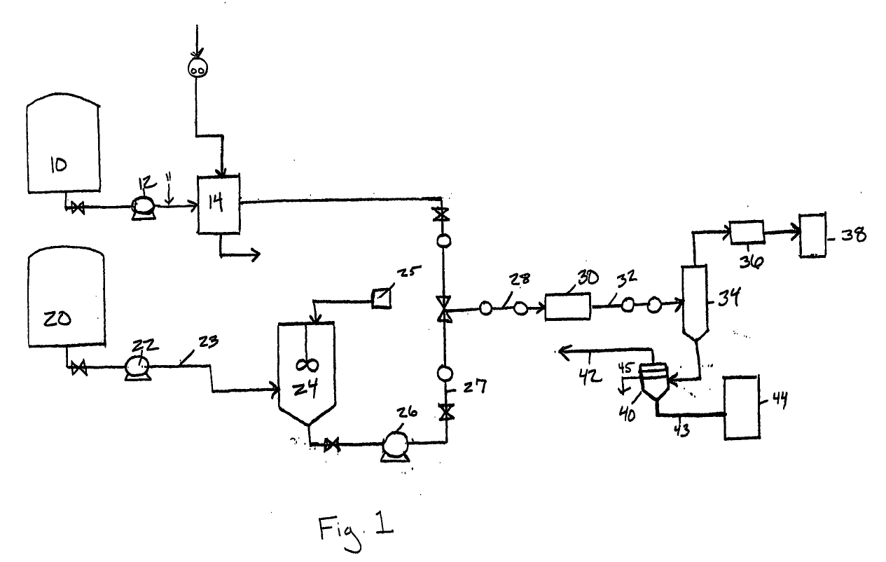

In greater detail, the present disclosure, including FIGS. 1-4 in which like numbers represent like parts throughout the several views, sets forth methods and apparatus for producing esters, particularly lower alkyl esters, such as fatty acid alkyl esters that are suitable for use as biodiesel. However, the presented methods and apparatus also can encompass production of other esters.

Among the aspects to which this disclosure is directed is the production of esters for use as biodiesel. Fatty acid alkyl esters used as biodiesel generally are produced during either esterification or transesterification reactions of biologically derived feed stocks.

Biodiesel generally is produced from the transesterification of a triglyceride with an alcohol in the presence of a catalyst. This transesterification reaction can be represented as follows:

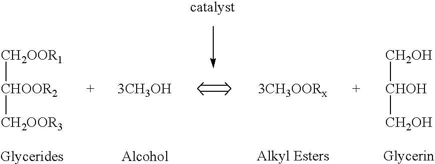

Alkyl esters suitable for production of biodiesel and other ester products can be produced using a variety of alcohols and catalysts. The alkyl esters can be derived from di- and mono-glycerides that are typically found in biologically derived oils. As used herein, the term “oil” refers to any biologically derived source of lipids that can undergo an esterification or transesterification reaction to form an ester. The term “oil” encompasses any biologically derived source of tri-, di-, or mono-acylglycerols however substituted. The term “oil” can encompass, but is not limited to, beef tallow; pork fat; poultry fat; oil from soybeans, cottonseeds, canola, rapeseeds, rice bran, flax seeds, safflowers, cranbe, corn, sunflowers, mustard seeds, palm, peanuts, coconuts, or other vegetable or animal material; used or recycled animal or vegetable oils; other biologically derived oils; and combinations thereof.

The alcohol employed to react with the oil can be any suitable alcohol or blend of alcohols for carrying out the reaction by which the ester is produced. For example, the alcohol can include one or more monovalent or multivalent alcohols, such as methanol, ethanol, isopropanol, butanol, trimethylpropane, glycerols and other polyols or combinations thereof.

The catalyst used to produce the ester can include any suitable acid or base. The catalyst can include a suitable base, such as, for example, sodium hydroxide, potassium hydroxide, and/or a suitable alkoxide such as sodium methoxide, potassium methoxide, sodium tert-butoxide, potassium tert-butoxide, magnesium ethoxide, barium ispropoxide, sodium isopropoxide, sodium methylate, potassium methylate and combinations thereof. Alternatively, the reaction can be carried out using an acid such as hydrochloric acid, sulfuric acid, phosphoric acid, nitric acid, other inorganic acids and combinations thereof.

The methods set forth herein generally encompass contacting reactants in a reaction zone during a mean residence time less than those mean residence times generally known in the art. As used herein, the term “mean residence time”, or space time, of the reactor or reaction zone is equal to the volume of the reactor or reaction zone divided by the volumetric flow rate entering such reactor or reaction zone. The methods can encompass both batch, semi-continuous and continuous reactors and reaction zones, but are particularly suited for continuous reactors and reaction zones. As used herein, the term “continuous” refers to the simultaneous input of reactants and output of products and/or reactants from a reactor or reaction zone. Furthermore, “continuous” can be used to describe a system wherein the reactants and/or products of the system are not divided into batches prior to entering or immediately after they exit the reactor or separation units of the system.

The methods and apparatus disclosed herein can encompass dynamic reactors in which the reactants are contacted to produce esters. As used herein, the term “dynamic reactor” encompasses a device, unit or portion thereof containing at least a portion of a reaction zone in which reactants can undergo esterification or transesterification reactions, such as the reactions set forth herein, and which comprises one or more moving rotating parts at least one of which contacts the fluid media containing the reactants and which are provided to mix mechanically the fluid media by such contact. The term “dynamic reactor” encompasses such devices that can provide sufficient shear, cavitation, and/or other forces to provide sufficient mixing of the reactant mixture necessary to carry out esterification and/or transesterification reactions within the ranges of mean residence times set forth herein. The term “dynamic reactor” can include a fluid contacting moving part such as an impeller or rotor. The “dynamic reactor” can include a motor operably connected to an impeller or rotor to provide motion to such moving part. In particular, the motor can be operably connected to the impeller or rotor by a shaft to allow the impeller or rotor to revolve within the fluid media.

The dynamic reactor disclosed in the methods and apparatus herein can include one or more colloid mills or pipeline mixers that can provide sufficient mixing to the reactants to allow for the reactions to occur within the ranges of mean residence times set forth herein. Examples of colloid mills include those offered by Chemicolloid Laboratories Inc., Garden City Park, N.Y. or Chemineer, Inc., Dayton, Ohio, or set forth in U.S. Pat. Nos. 6,745,961; 6,305,626 the disclosures of which are hereby incorporated by reference as if set forth in their entirety herein. Examples of pipeline mixers include those offered by Chemineer, Inc., Dayton, Ohio or set forth in U.S. Pat. No. 4,066,246 the disclosure of which is hereby incorporated by reference as if set forth in its entirety herein.

The dynamic reactors disclosed herein also may include one or more cavitation reactors or mixers that can provide sufficient mixing to carry out esterification or tranesterification reactions, such as those disclosed herein, within the range of mean residence times set forth herein. A cavitation reactor induces cavitation in fluid media containing reactants. Cavitation, sometimes referred to as hydrodynamic cavitation, is a phenomenon in which cavities or cavitation bubbles filled with a gas or voids form either inside a fluid flow or at the surface of a body, such as a rotor, propeller, or impeller, in contact with the fluid flow resulting from a local pressure drop in the fluid. The number of such cavities or bubbles can be large depending upon the pressure distribution across the fluid flow. As the cavitation bubbles are exposed to increased pressures within the fluid flow, they rapidly collapse, thereby generating localized pressure impulses having internal pressures that can reach 150,000 psi. The result of these high-pressure implosions is the formation of shock waves that emanate from the point of each collapsed bubble. Such high-impact loads tend to disperse or separate media adjacent the collapsing bubbles, thereby leading to more intense mixing of the neighboring fluids than would otherwise be attained. Examples of cavitation reactors include the Cavitron line of reactors offered by Arde-Barinco, Inc. of Norwood, N.J., as well as those set forth in U.S. Pat. Nos. 6,935,770; 6,910,448; 6,857,774; 6,627,784; 6,386,751; 5,957,122; 5,522,553; 5,188,090; 3,791,349; and 3,242,908 the disclosures of which are hereby incorporated by reference as if set forth in their entirety herein.

The dynamic reactor can include a combination of two or more of such colloid mills, pipeline mixers and/or cavitation mixers.

As shown in FIG. 1, the apparatus or system for producing esters, such as lower alkyl esters, includes an oil storage tank 10, which contains oil as described herein. The oil storage tank 10 is in fluid communication with a pump 12 that is used to transfer the oil to at least to the next unit in the system. Indeed, the system disclosed herein can include multiple oil storage tanks, each of which can contain a particular type of oil or oil mixture. These tanks can be interconnected with the other process units of the system to provide optional feedstock compositions to the downstream reactor unit. For example, one oil storage tank may contain poultry fat, another soybean oil and a third used cooking oil or “yellow grease”. These tanks can be selectively opened to the remainder of the system to allow for the production from these various feedstocks to produce lower alkyl esters with varying characteristics.

The oil storage tank 10 is in fluid communication with a heat exchanger 14 that supplies heat to the oil as it moves through the system. The oil stored in tank 10 can be directed using pump 12 through pipe 11 to heat exchanger 14. The temperature of the oil can be raised by the heat exchanger 14 to a predetermined temperature, which may be the temperature of the reaction by which alkyl esters are formed or some intermediate temperature. For example, the oil temperature can be adjusted to within a range of approximately 30° C. to approximately 200° C. In another aspect, the oil temperature can be adjusted to within a range of approximately 45° C. to approximately 100° C., and, in yet another aspect, the temperature of the oil can be adjusted to within a range of approximately 50° C. to approximately 85° C. Alternative temperature range within these ranges are also contemplated. Alternatively, the oil storage tank 10 can be supplied with heat so as to maintain the oil temperature within a desired range prior to directing the oil through pipe 11.

Alcohol storage tank 20 contains an alcohol or alcohol mixture as described above which is to be combined with the oil from the storage tank 10 in the production of alkyl esters. The alcohol storage tank 20 is operably connected to a pump 22 and is in fluid communication through pipe 23 with a mixing vessel 24. Alcohol is delivered through pipe 23 to the mixing vessel 24, wherein a catalyst as described above is combined with the alcohol. The amount of alcohol used depends upon the composition and molecular weight of the alcohol, as well as the composition and molecular weight of the oil. In one aspect, the theoretical amount of alcohol used can be about 3 moles alcohol to one mole triglyceride. In another aspect, the amount of alcohol used can be in a range of about 2.5 to about 8 mole alcohol/mole triglyceride. In another aspect, the amount of alcohol can be in a range of about 3.5 to about 6.5 mole alcohol/mole triglyceride. In a further aspect, the amount of alcohol can be in a range of about 4 to about 6 mole alcohol/mole triglyceride.

The catalyst can be charged to the mixing vessel 24 by a fluid or solids delivery unit 25 depending upon the state of the catalyst. The amount of catalyst added to the mixing vessel is predetermined based on the free fatty acid content of the oil and the average molecular weight of the oil to be reacted. Furthermore, the composition and molecular weight of the catalyst affects the amount of catalyst used. The amount of catalyst can be in the range of about 0.1% to about 2% by weight, additionally the catalyst may be in the range of about 0.2% to about 1.5% by weight, and furthermore, may be in the range of about 0.25% to about 1.0% by weight.

The alcohol and catalyst are mixed in the mixing vessel 24 and are then directed by pump 26 or otherwise through pipe 27. Likewise, oil is directed through pipe 17 to pipe 28 where it then combines with alcohol from pipe 27.

The alcohol, catalyst and oil are then directed into the dynamic reactor 30 wherein they react to produce lower alkyl esters and glycerol. The dynamic reactor 30 imparts a high degree of mixing in a relatively short time period thereby allowing for mean residence times in the reaction zone to be significantly shorter than those typically found in the production of fatty acid methyl esters found in biodiesel. Instead of measuring mean residence times in hours, they can be measured in minutes or seconds with the methods and apparatus disclosed herein. For example, the mean residence time can be less than ten minutes. In another aspect, the mean residence time can be less than five minutes. In yet another aspect, the mean residence time can be less than two minutes. In still a further aspect, the mean residence time can be less than one minute. In yet another aspect, the mean residence time can be less than 30 seconds. In still a further aspect, the mean residence time can be less than ten seconds. In another aspect, the mean residence time can be in a range of about 3 to about 15 seconds. In a further aspect, the mean residence time can be in a range of about 5 to about 10 seconds.

The mean residence time required to carry out the transesterification of the oil is a function of the temperature, pressure, ratio of reactants, and degree of mixing. In the methods and apparatus of the present disclosure, the inlet and outlet pressures of the fluids into and out of the dynamic reactor can be within a range of about 1 atm to about 5 atm. In another aspect, the pressures can be in a range of about 1.5 atm to about 3 atm. By providing increased shear and/or cavitation and/or energy to the reactants in the reaction zone, the transesterification reaction can approach completion and/or equilibrium more rapidly, thereby reducing the mean residence time required to carry out the reaction. The dynamic reactor can provide heat to the reactant mixture so as to provide a temperature differential between the inlet temperature of the reactants and the outlet temperature of the products. The temperature differential between the inlet and outlet flows of the dynamic reactor can be in the range of 0° C. to about 30° C. In yet another aspect, the temperature differential can be in the range of about 5° C. to about 15° C. In still a further aspect, the temperature differential can be in the range of about 6° C. to about 10° C.

A vapor-liquid separator 34 is in fluid communication with the dynamic reactor 30. The product mixture, which can contain lower alkyl esters, glycerol, unreacted oil, alcohol, catalyst and byproducts and which can include both a liquid component and a vapor component, exits the first reactor 30 through the pipe 32 and enters the first vapor-liquid separator 34. Vapor contained in the reaction mixture exits the upper portion of the vapor liquid separator 34, and is condensed in condenser 36. The condensed vapor is then collected in recovery tank 38, which is in fluid communication with the condenser 36. As shown in FIG. 1, the entire product mixture stream (both liquid and vapor) exiting the dynamic reactor 30 enters the vapor-liquid separator 34.

As shown in FIG. 1, the entire liquid product mixture exits from the lower portion of the vapor-liquid separator 34 and enters separator 40. The separator 40 can be a liquid-liquid separator, such as a centrifuge, which separates the components of the product mixture by centrifugal fractionization into a light liquid phase and a heavy liquid phase based on the specific gravity of the components. Depending upon the composition of the product mixture, the separator 40 can be a liquid-liquid-solid separator, such as a desludge type centrifuge that separates the product mixture into a light liquid phase, a heavy liquid phase, and a solid or semi-solid phase. The light liquid phase exits the separator 40 through pipe 42 and the heavy liquid phase, or bottoms, exits through pipe 43. The heavy liquid phase is collected in a bottoms storage tank 44 for further processing. Any solids separated from the light and heavy liquid phases can be removed from the separator 40 through pipe 45.

The light liquid phase contains primarily alkyl esters and can be directed through pipe 42 for further processing as necessary.

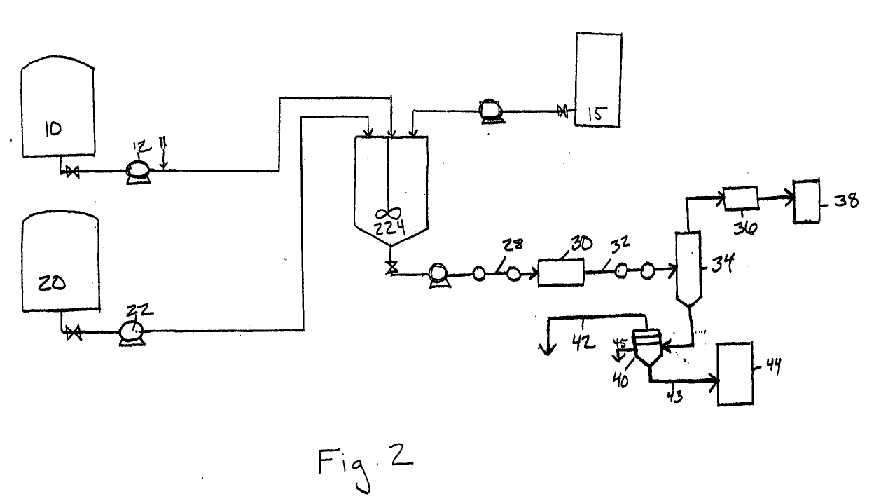

In FIG. 2, the oil fed from oil storage tank 10, the alcohol fed from storage tank 20 and the catalyst fed from storage container 15 are fed into a mixing unit 224, which can be a stirred tank. The reactants are fed in the correct ratios to the mixing unit 224, where they optionally can be heated to the predetermined temperatures set forth above. The reactants are mixed to form a reactant mixture that is then fed to the dynamic reactor 30 through pipe 28. As with the system set forth in FIG. 1, the reactant mixture is introduced into a reaction zone within the dynamic reactor 30 where the mixture reacts to produce a product mixture that contains a majority by weight of lower alkyl esters. The product mixture then exits the dynamic reactor 30 and can be separated as described above.

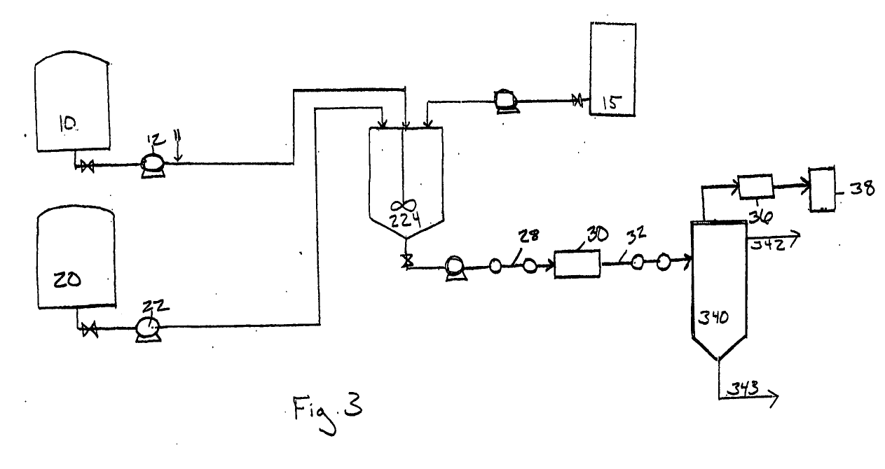

FIG. 3 discloses another system in which lower alkyl esters can be produced by the reaction of an oil, an alcohol and a catalyst in a dynamic reactor with mean residence times as set forth above. The reactant mixture entering the dynamic reactor 30 is formed as described in relation to the system shown in FIG. 2. Unlike that system, however, the product mixture exiting the dynamic reactor 30 is fed through pipe 32 to a separator 340, which can be a tank, decanter or similar unit in which the constituents of the product mixture separate by gravity. The vapor component of the product mixture enters the condenser 36 from separator 340. The heavy phase liquid, which can contain glycerol, unreacted alcohol, catalyst, free fatty acids, soaps, salts, and emulsified esters, is removed from the lower portion of the separator 340 through pipe 343. The light liquid phase, containing primarily lower alkyl esters is removed from the upper portion of the separator 340.

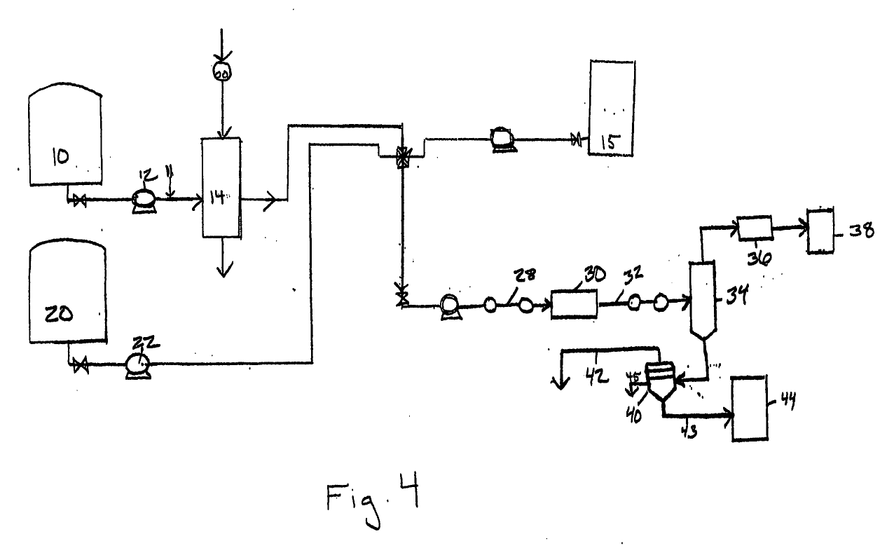

In FIG. 4, the oil, the alcohol and the catalyst are introduced from the respective oil storage tank 10, the alcohol storage tank 20 and the catalyst storage container 15 individually into the dynamic reactor 30. These reactants can be combined directly in the dynamic reactor to form a reaction mixture or just upstream of the inlet of the dynamic reactor 30. A heat exchanger 14 can adjust the temperature of the oil to a predetermined temperature, such as into the range of the reaction temperature prior to entering the dynamic reactor. Alternatively, the temperature of the reaction mixture can be adjusted to the appropriate reaction temperature within the dynamic reactor.

In each of the apparatus set forth in FIGS. 1-4, the oil, alcohol and catalyst, as well as the reactant mixture which they form generally is substantially free of lower alkyl esters as they enter the dynamic reactor and/or cavitation zone of the system. The lower alkyl esters are produced within the dynamic reactor and are discharged for further processing. Alternatively, when a side stream containing lower alkyl esters from the outlet of the separator is fed into the inlet of the dynamic reactor, the reactant mixture can contain some lower alkyl esters. However, such alkyl esters will have been produced within the dynamic reactor and not prior to the initial introduction of the material entering the reaction zone of the system.

The present disclosure encompasses reconfigurations of the systems and dynamic reactors disclosed herein.

It will be understood by those skilled in the art that while various embodiments and examples have been discussed herein various additions, modifications and changes can be made thereto without departing from the spirit and scope of the disclosure.

1-62. (canceled)

63. A method for producing a lower alkyl ester comprising:
introducing an oil, an alcohol and a catalyst into a dynamic reactor to form a reactant mixture, wherein the reactant mixture is substantially free of lower alkyl esters;
reacting the reactant mixture in the dynamic reactor to produce a product mixture, wherein the product mixture comprises a majority by volume of lower alkyl esters, and wherein the mean residence time of the dynamic reactor is less than about ten seconds.

64. The method of claim 63, further comprising inducing cavitation within the dynamic reactor during reacting.

65. The method of claim 64, wherein inducing cavitation within the dynamic reactor comprises revolving a rotor.

66. The method of claim 63, wherein the product mixture comprises a majority by weight lower alkyl esters.

67. The method of claim 66, wherein the product mixture comprises at least about 80% by weight of lower alkyl esters.

68. The method of claim 63, further comprising separating the product mixture into a vapor product phase and a liquid product phase.

69. The method of claim 63, further comprising separating the product mixture into a light liquid phase and a heavy liquid phase, wherein the light liquid phase comprises a majority by volume of lower alkyl esters.

70. The method of claim 69, wherein separating comprises centrifugally separating the product mixture.

71. A method for producing lower alkyl esters comprising:
introducing reactant mixture into a cavitation zone, wherein the reactant mixture is substantially free of lower alkyl esters;
reacting the reactant mixture within the cavitation zone to produce a product mixture, wherein the mean residence time within the cavitation zone is less than about ten seconds; and,
separating the product mixture into a light liquid phase and a heavy liquid phase, wherein the light liquid phase contains a majority by volume of lower alkyl esters.

72. The method of claim 71, further comprising removing vapor from the product mixture prior to separating.

73. The method of claim 71, wherein separating comprises centrifugally separating the product mixture into the light liquid phase and the heavy liquid phase.

74. The method of claim 71, wherein the light liquid phase comprises at least about 90% by weight lower alkyl esters.

75. The method of claim 71, further comprising inducing cavitation within the cavitation zone by revolving a rotor.

76. The method of claim 71, wherein the mean residence time is less than about five seconds.

77. A method for producing a fatty acid methyl ester comprising:
combining an oil, methanol and a base catalyst to form a reaction mixture, wherein the reaction mixture is substantially free of fatty acid methyl esters;
reacting the reaction mixture in a dynamic reactor during a mean residence time of less than about 10 seconds to form a product mixture;
separating the product mixture into a light liquid phase and a heavy liquid phase, wherein the light liquid phase contains a majority by weight of fatty acid methyl esters.

78. The method of claim 77, wherein reacting comprises inducing cavitation within the reactant mixture.

79. The method of claim 78, wherein inducing cavitation comprises revolving a rotor.

80. The method of claim 77, further comprising removing vapor from the product mixture prior to separating the product mixture into a light liquid phase and a heavy liquid phase.

81. The method of claim 77, wherein the light liquid phase contains a majority by weight of fatty acid methyl esters.

82. The method of claim 77, wherein the mean residence time is less than about five seconds.
